(19) United States
(12) Patent Application Publication
(10) Pub. No.: US 2004/0111022 A1
(43) Pub. Date: Jun. 10, 2004

(54) Intrapericardial MRI device and method

(76) Inventors: James R. Grabek, Minneapolis, MN (US); Michael F. Hoey, Shoreview, MN (US)

Correspondence Address: Beck & Tysver, P.L.L.C., Suite 100, 2900 Thomas Avenue S., Minneapolis, MN, 55416, US

(21) Appl. No.: 10/641,474

(22) Filed: Aug. 15, 2003

Related U.S. Application Data
(60) Provisional application No. 60/403,982, filed on Aug. 16, 2002.

(51) Int. Cl. A61B 5/05

(52) U.S. Cl. 600/423

(57) ABSTRACT

A catheter based loop antenna is delivered to the pericardial space through an opening in the chest. The size of the antenna may be modified to selectively view tissue for imaging or spectrographic analysis purposes.

### CROSS REFERENCE

The Applicant claims the benefit and incorporates by reference U.S. Provisional Patent No. 60/403,982, filed Aug. 16, 2002, titled “Collapsible Loop Antenna for In Vivo Magnetic Resonance”.

### FIELD OF THE INVENTION

The present invention relates generally to magnetic resonance (MR) imaging and MR spectroscopy of living tissue and more particularly to an antenna for minimally invasive surgical use.

### BACKGROUND OF THE INVENTION

Advances in magnetic resonance imaging have placed this non-invasive imaging technology at the forefront of medical imaging technologies. Although MRI imaging is widely used in a variety of diagnostic settings, it is rarely used to image small regions of the body. Although the use of MRI devices for spectroscopy and thermal measurements these applications are not widely practiced.

Some examples of small region imaging technologies are taught by U.S. Pat. No. 5,964,705 to Truwit which shows a solenoid antenna coil mounted on the distal tip of a intravascular catheter. This approach allows one to image the vessel walls as a mechanism for ascertaining the underlying disease-state. This intravascular use is minimally invasive but suffers from a number of limitations.

There is a continuing need to improve in vivo MRI devices and techniques for in vivo use.

### SUMMARY OF THE INVENTION

In contrast to the prior art the present invention provides a collapsible loop MRI antenna which can be introduced into the pericardial space surrounding the patient's heart. The diameter of the loop can be adjusted while in situ and it can be used to image relatively large areas and relatively small areas. This advantage permits the device to be used to locate vessels or other regions of interest than to navigate to those regions and adjust the antenna size so that the resolution of the image is sufficient for the diagnostic purpose.

The ability to restrict or expand the field of view for imaging also permits the device to be used quantitatively and qualitatively for spectrographic analysis of suspected lesions and the like.

The ability to monitor the composition of lesions within the heart as well as image them allows a differential diagnosis of a lesion between vulnerable plaque and other disease states. The device can also be used to measure the temperature of tissue as an aid to distinguishing lesions from each other and may especially useful to determine the degree of inflammation of vulnerable plaque.

The MRI antenna may also be used to follow the course of RF ablation applied to the heart wall from either the pericardial space or from the blood pool within the heart. The present invention relates to the use of a small collapsible loop antenna having a nominal diameter between one and five centimeters. The coil is deployed in the pericardial space using a PerDUCER access approach.

Once inside the pericardial space the loop antenna is navigated to the coronary arteries where it may be positioned over sections of the coronary artery. Since the loop antenna and the heart are moving it is likely that there will be minimal artifacts associated with the motion of the antenna and this will allow higher resolution imaging and spectroscopy of the coronary artery sites. In addition to spectrographic analysis or imaging analysis to characterize the nature of the plaque deposits, it is also possible to measure the temperature of the plaque departments using the MR antenna. This technique relies on the detection of the brownie in motion of the molecules based upon their temperature. It is expected that temperature differences as small as a few tenths of a degree can be detected, imaged and presented to the physician to help characterize the nature of the plaque.

### BRIEF DESCRIPTION OF THE DRAWINGS

Throughout the several figures identical refers to identical structure wherein:

FIG. 1 is a schematic over view of the device;

FIG. 2 is a partial view depicting the distal tip of the device;

FIG. 3 is a cross section of a portion of the device;

FIG. 4 is a first embodiment of the antenna;

FIG. 5 is a second embodiment of the antenna;

FIG. 6 is a second embodiment of the antenna;

FIG. 7 is a second embodiment of the antenna;

FIG. 8 is a third embodiment of the antenna;

FIG. 9 is a third embodiment of the antenna;

FIG. 10 is a panel depicting a step in a method;

FIG. 11 is a panel depicting a step in a method;

FIG. 12 is a panel depicting a step in a method;

FIG. 13 is a panel depicting a step in a method;

FIG. 14 is a panel depicting a step in a method;

FIG. 15 is a panel depicting a step in a method;

FIG. 16 is a panel depicting a step in a method; and,

FIG. 17 is a panel depicting a step in a method.

### DETAILED DESCRIPTION

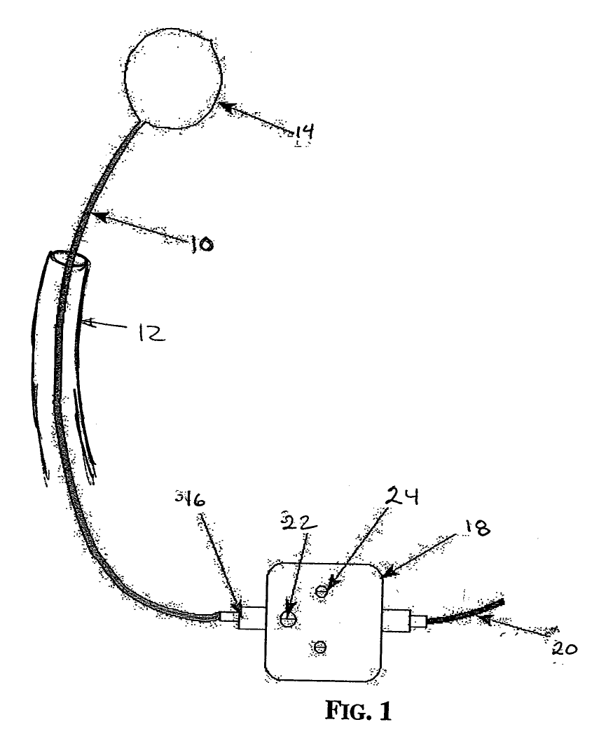

FIG. 1 shows the antenna device 10 positioned within an intrapericardial access sheath 12. The distal tip of the device 10 is formed as a loop 14. The proximal end of the device 10 terminates in a proximal connector 16, which is coupled to a matching network 18. The matching network in turn is connected to the MRI machine through a cable 20. The function of the matching network is to match the impedance of the loop 14 with the required impedance of the MRI machine. This may be done automatically or through manual adjustments shown in the figure as adjustment screw 22 and 24. In general the nature of matching networks is well known in this art and an LRC network will be provided to tune the antenna to the MRI machine.

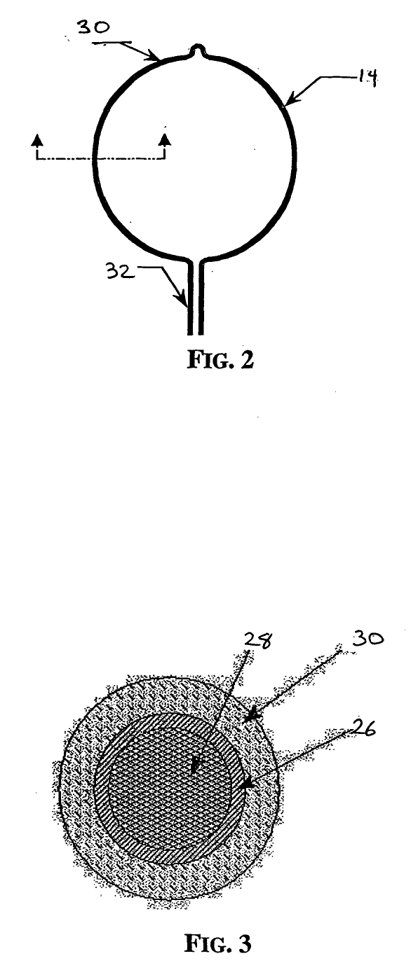

FIG. 2 and FIG. 3 show the distal tip loop 14 in more detail. FIG. 2 depicts the unconstrained shape of the device forming a circular loop antenna as opposed to other shapes. A biocompatible surface coating 30 is applied to the underlying substrate 28. FIG. 3 shows a cross section of the loop14. It is preferred to form the underlying substrate material from nitinol with a preferred conductivity coating 26 of gold. A biocompatible insulated sheath is formed over the individual wire elements as indicated by insulation 30. As shown in the figure, the loop antenna terminates in a twin line transmission line 32. Each leg of this line may be individually manipulated and the spacing between the legs is retained at a constant distance to prevent impedance mismatching.

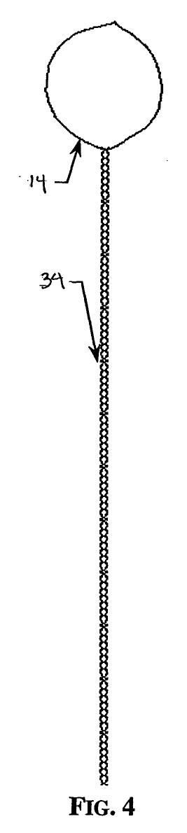

As an alternative to the twin line transmission line depicted in FIG. 2 and FIG. 6, a twisted pair transmission line 34 may be used to couple the loop 14 to the matching network as seen in FIG. 4. In FIG. 5 an external insulating sheath 36 is supplied over the transmission line and the interior cross-section of the transmission line may be an insulated twin line construction shown in FIG. 6 with a nitinol core 38 surrounded by a gold sputtered coating 40, which is held together at a fixed distance from the other conductor.

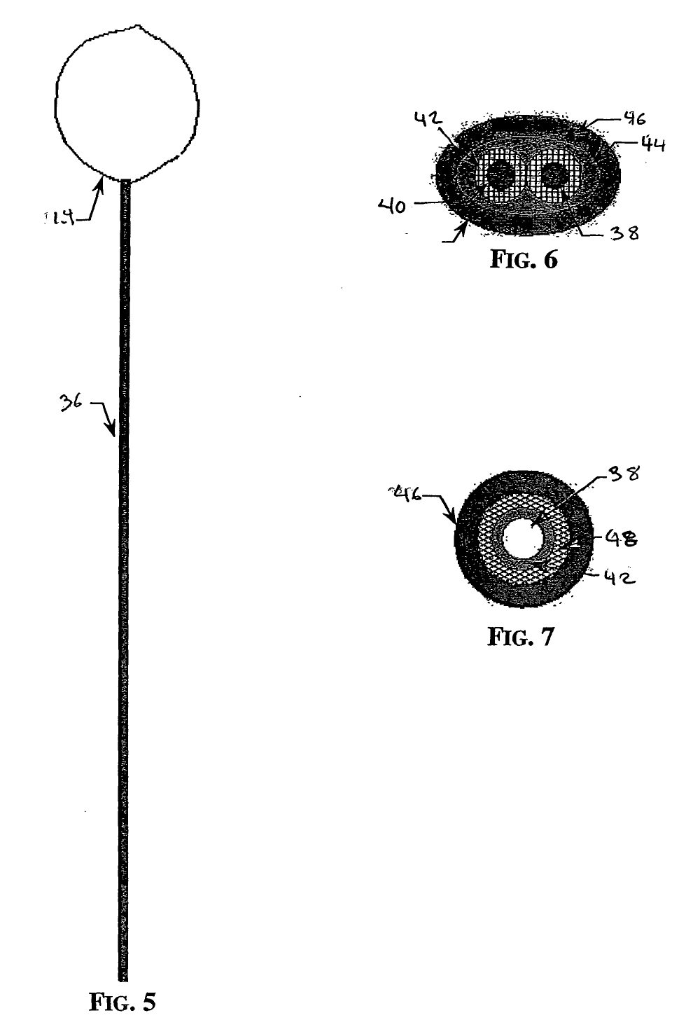

As an alternative a coaxial construction may be adopted as seen in FIG. 7 where the exterior insulating layer 46 is coaxial with the nitinol substrate, once again coated with a conductivity enhancing coating such as gold 42. A braid 48 may be provided to provide electrical connection for the ground reference of the loop antenna 14.

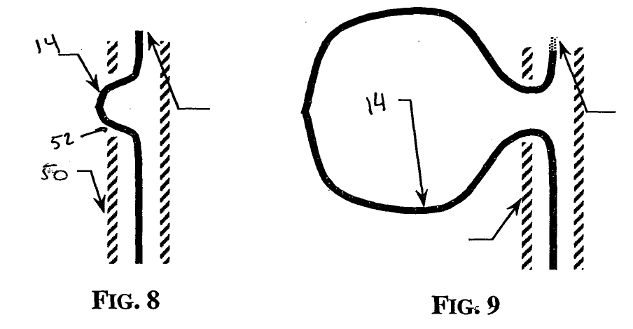

FIG. 8 and FIG. 9 should be considered together. FIG. 8 shows an alternative form of construction where a nitinol loop 14 is delivered out of the side port of a catheter 50 through an aperture 52. As the loop emerges as seen in FIG. 9 the shape memory property of the nitinol core forms a circular loop. Each leg is connected to the MRI matching network through connections not shown in FIG. 9.

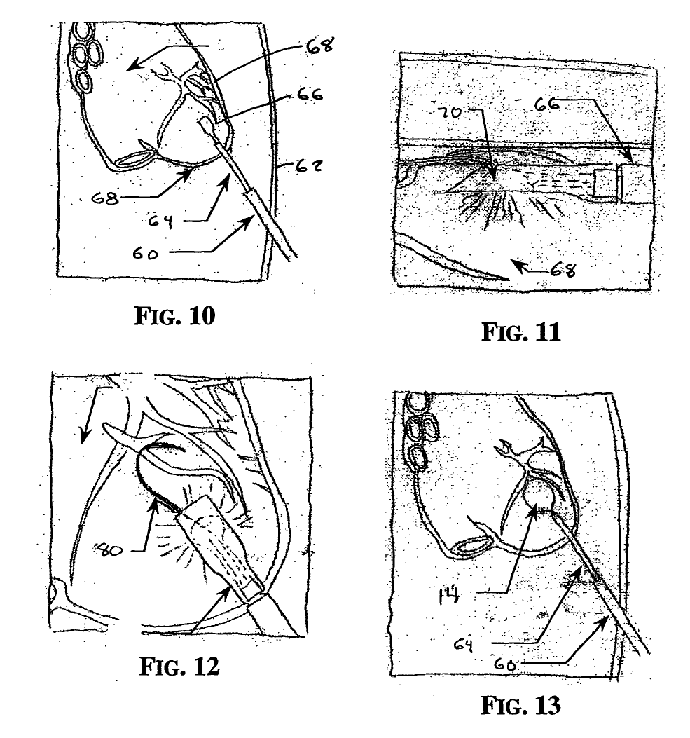

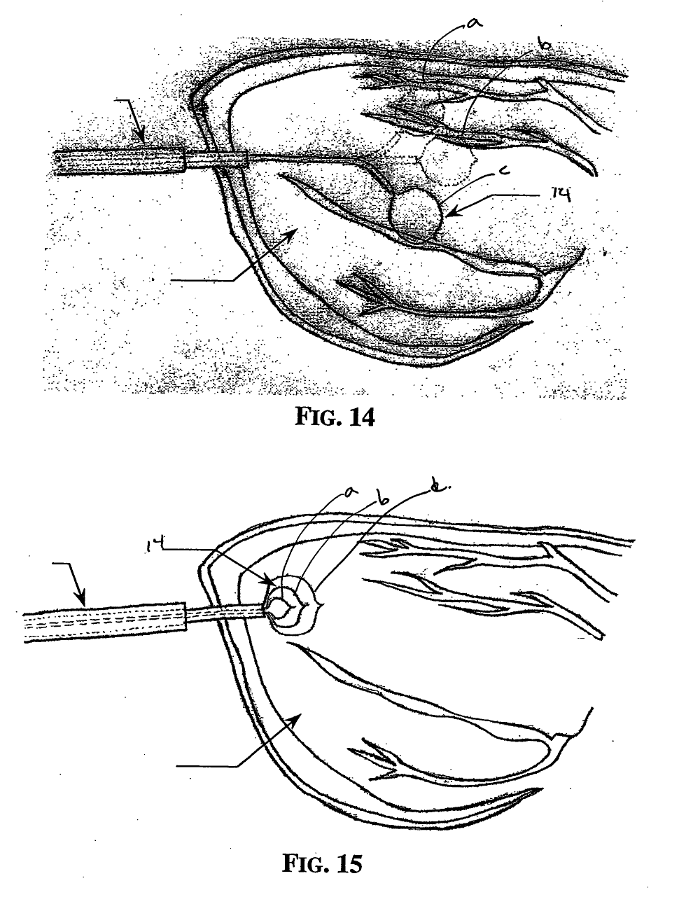

FIG. 10 shows a step in the method of introducing the pericardial MRI antenna into the pericardial space through the use of a PerDUCER device as manufactured by Comedicus of Minneapolis, Minn. In FIG. 10 the PerDUCER device has been inserted through the chest wall 62 and advanced to the pericardial “sac”. A procedure sheath 64 allows the PerDUCER 66 to approach the pericardial space of the heart while leaving the pericardium 68 intact. The distal tip of the PerDUCER 66 includes a bleeb forming suction device 70 which draws the pericardium 68 into the device permitting it to be pierced as seen in FIG. 11.

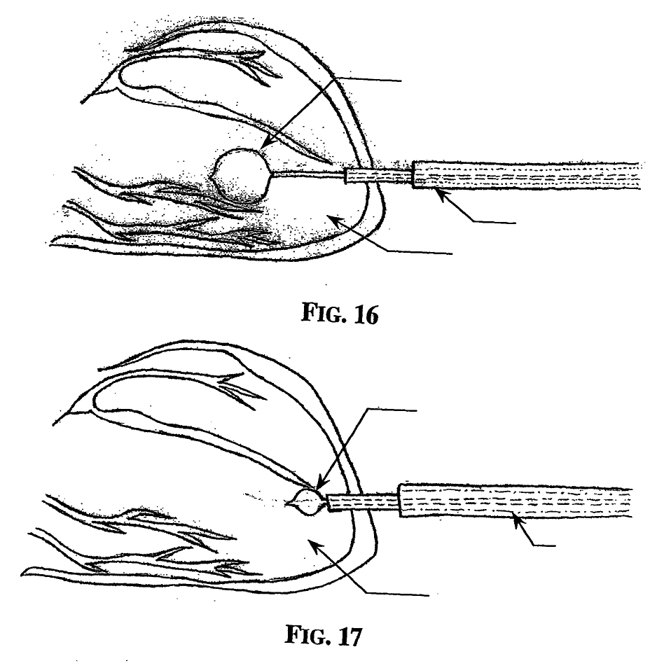

FIG. 12 shows a guidewire 80 being deployed through the hole in the pericardial sac permitting the entry of other devices into the pericardial space such as the MRI antenna introduced through sheath 60 and sheath 64. As seen in FIG. 14 the loop 14 may be manipulated to multiple positions indicated with reference numeral a, b and c in the figure. With the loop deployed into its maximum diameter configuration imaging can be performed helping the physician locate anatomic features of interest such as the coronary arteries. FIG. 14 shows the loop being adjusted to multiple diameters seen in the figure as diameter a, b and c. The imaging field of view depends directly upon the diameter of the device. When operated in a spectrographic mode where the underlying physiology is measured by spectroscopy the smaller the loop the smaller volume is interrogated. In FIG. 15 for example, the physician may be reducing the size of the loop antenna from position c to position a to interrogate whether or not a particular underlying piece of cardiac tissue is ischemic. In FIG. 16 a coronary artery is approached as seen in FIG. 17 and the loop of the antenna is reduced to provide both imaging and spectrographic analysis of the nature of the lesion present there. It is believed that this technique of imaging along with spectroscopy can allow the identification of vulnerable plaque. When the loop is small it is possible to monitor the temperature of tissue using the MRI system and it is a portion of the method of this invention to provide both imaging, spectrographic and temperature measurement capabilities in a single antenna device placed over a single location of the heart with the data taken at the same time, or sequentially without moving the loop.

With regard to FIG. 14 it should be clear that the physician may be performing an RF ablation procedure on the interior of the heart. In this instance the pericardial loop antenna can be used to “track” the therapeutic lesion by imaging, thermal sensing or spectrographically. Although not illustrated in the FIG. 1f the ablation procedure is performed in the pericardial space then the MRI antenna can be deployed inside the heat to rack the procedure.

What is claimed:

1 An MRI antenna device for connection to an MRI machine comprising:
an elongate body having a proximal end and a distal end;
a sheath positioned over said elongate body;
a loop antenna having a substantially circular shape formed from a super elastic material; whereby retraction of said loop can be retracted into said sheath, or advanced out of said sheath thereby forming said circular shape;
a transmission line coupled to said loop and extending to said proximal end of said elongate body;
a connector coupled to said transmission line at said proximal end.

2. A method of diagnosing the human body with the device of claim 1 comprising;
introducing the device into the pericardial space;
altering the size of the circular shape of the loop to image tissue thereby locating a position of interest;
reducing the size of the loop to a small size to interrogate and evaluate the position of interest spectrographically.

3. A method of diagnosing the human body with the device of claim 1 comprising;
introducing the device into the pericardial space;
altering the size of the circular shape of the loop to image tissue thereby locating a position of interest;
reducing the size of the loop to a small size to interrogate and evaluate the position of interest thermally.
